(19) United States
(12) Patent Application Publication
(10) Pub. No.: US 2024/0277761 A1
(43) Pub. Date: Aug. 22, 2024

(54) HUMAN IPSC-DERIVED MACROPHAGES FOR LIVER REPAIR AND REGENERATION

(71) Applicant: The Regents of the University of California, Oakland, CA (US)

(72) Inventors: Dan S. Kaufman, San Diego, CA (US); Somayeh Pouyanfard, San Diego, CA (US); Tatiana Kisseleva, La Jolla, CA (US)

(21) Appl. No.: 18/580,524

(22) PCT Filed: Jul. 15, 2022
(86) PCT No.: PCT/US2022/037306

§ 371 Date: Jan. 18, 2024

Related U.S. Application Data
(60) Provisional application No. 63/224,318, filed on Jul. 21, 2021.

(51) Int. Cl. A61K 35/15; A61P 1/16; C12N 5/0786

(52) U.S. Cl. CPC A61K 35/15; A61P 1/16; C12N 5/0645; C12N 2501/2304; C12N 2501/2313; C12N 2501/24; C12N 2506/45

(57) ABSTRACT

Compositions, methods of treatment for liver disease such as liver fibrosis, compositions, and methods for the manufacture thereof, comprising a plurality of macrophages derived from human induced pluripotent stem cells (iPSCs), wherein the macrophages are polarized to a pro-inflammatory M1 phenotype and/or an anti-inflammatory M2 phenotype. Administration reduces fibrogenic gene expression and liver disease associated histological markers. The M1 macrophages express elevated CD80, TNF-α and IL-6. The M2 macrophages express elevated CD206, CCL17, and CCL22.

### CROSS-REFERENCE TO RELATED APPLICATIONS

This application claims priority to U.S. Provisional Patent Application Ser. No. 63/224,318 filed Jul. 21, 2021, the entire contents of which are incorporated herein by reference.

### TECHNICAL FIELD

The present invention relates to engineered immunotherapies.

### BACKGROUND

Chronic toxic liver injury due to various etiologies including viral infection (hepatitis B and C), metabolic disorders (non-alcoholic steatohepatitis), exposure to chemicals (alcoholic liver diseases) or autoimmune diseases (autoimmune hepatitis) can lead to the development of liver fibrosis and cirrhosis¹. Liver injury leads to hepatocyte apoptosis, recruitment of inflammatory cells, and activation of myofibroblasts which secrete extracellular matrix (ECM) proteins and result in a fibrous scar. Liver fibrosis progresses to cirrhosis, hepatocellular carcinoma, and eventual liver failure² where organ transplantation is the only treatment option³. An alternative therapy is transplantation of cells that enable liver repair and/or regeneration. Transplantation of myeloid cells alone^{4,5} or in combination with mesenchymal stem/stromal cells (MSCs)⁶ have been shown to suppress inflammatory responses, and as a result, lead to the improvement of liver function. Infusion of autologous macrophages in patients with end stage liver disease⁷. Several other cell types have shown efficacy in preclinical models, including hepatocytes⁸, liver sinusoidal endothelial cells⁹, and endothelial progenitor cells¹⁰.

Watanabe et. al.¹¹ demonstrated that combination of MSCs and macrophages had a synergistic effect and reduced liver fibrosis and enhanced liver function in mice with carbon tetrachloride (CCI₄)-induced fibrosis. This synergistic effect was attributed to the fact that MSCs induced macrophage polarization towards an M2 phenotype with high phagocytic capacity and increased expression of MMPs. Host macrophages and neutrophils were also shown to infiltrate the fibrotic region after combination therapy and contributed to liver fibrosis regression and promoted regeneration along with transplanted cells.

Another study investigated the regenerative function of murine bone marrow-derived macrophages (BMM) in a CCI₄-induced mouse model of liver fibrosis. Although the macrophages were not polarized to either M1 or M2, they found that intraportal injection of BMM led to an up-regulation of chemokines (MCP-1, MIP-1a, MIP-2) followed by hepatic recruitment of endogenous macrophages and neutrophils that delivered MMPs-13 and-9, respectively, to the scar site. There was a reduction in the number of hepatic myofibroblasts, and increased expression of the anti-inflammatory and tissue regenerative factors [(IL-10, TWEAK (TNFSF12) and oncostatin M (OSM)]. These studies demonstrate clinically relevant improvement of liver fibrosis upon macrophage treatment⁴.

Haideri et al. assessed the reparative function of murine embryonic stem cell (mESC)-derived macrophage in comparison to mouse BMM in a CCI₄-injury model. They found that mESC-derived macrophages were more skewed towards an anti-inflammatory phenotype, and therefore more able to reduce fibrosis compared to BMM. In these studies, mESC-derived macrophages reduced the amount of liver fibrosis to 50% of control treatment, decreased the number of fibrogenic myofibroblasts and activated liver progenitor cells¹².

Macrophages are of particular interest due to their intrinsic plasticity¹³. While classically activated M1 macrophages are known to drive inflammation and activation of hepatic stellate cells (HSCs) and myofibroblasts, M2 macrophages are able to alleviate inflammation and stimulate regeneration in the injured liver 14. Therefore, macrophage polarization to an anti-inflammatory M2 phenotype provides a novel strategy for treatment of liver fibrosis of different etiologies.

### SUMMARY OF THE INVENTION

The invention provides therapeutic cellular compositions and methods to generate macrophages from human induced pluripotent stem cells (iPSCs) and differentiate them to the M1 or M2 phenotype.

In embodiments, the invention provides methods to treat damaged liver tissue, comprising administering to a subject in need thereof an effective amount of a composition comprising a plurality of macrophages derived from human induced pluripotent stem cells (iPSCs), wherein the macrophages are polarized to a pro-inflammatory M1 phenotype and an anti-inflammatory M2 phenotype. In embodiments, the subject has liver fibrosis. In embodiments, the administration reduces fibrogenic gene expression and liver disease associated histological markers. In embodiments, the invention contemplates administering one or both M1 and M2 phenotypes.

In embodiments, the invention provides compositions comprising a plurality of macrophages derived from human induced pluripotent stem cells (iPSCs), wherein the macrophages are polarized to a pro-inflammatory M1 phenotype and an anti-inflammatory M2 phenotype. In embodiments, the M1 macrophages express elevated CD80, TNF-α and IL-6. In embodiments, the M2 macrophages express elevated CD206, CCL17, and CCL22.

In embodiments, the invention provides methods for the manufacture of a cellular composition comprising deriving a plurality of macrophages from human induced pluripotent stem cells (iPSCs), and polarizing the macrophages to a pro-inflammatory M1 phenotype and an anti-inflammatory M2 phenotype, thereby manufacturing the cellular composition. In embodiments, the macrophages are polarized to pro-inflammatory M1 in presence of LPS+IFN-γ. In embodiments, the M1 macrophages express elevated CD80, TNF-α and IL-6. In embodiments, the macrophages are polarized to anti-inflammatory M2 phenotypes in presence of IL-4+IL-13. In embodiments, the M2 macrophages express elevated CD206, CCL17, and CCL22.

In embodiments, the invention provides a pharmaceutically acceptable composition comprising a plurality of macrophages derived from human induced pluripotent stem cells (iPSCs), wherein the macrophages are polarized to a pro-inflammatory M1 phenotype and an anti-inflammatory M2 phenotype, as described herein.

### BRIEF DESCRIPTION OF THE DRAWINGS

FIGS. 1A-1F shows exemplary production of human iPSC-derived macrophages. FIGS. 1A-1B show a schematic diagram showing culture conditions required for each differentiation step from human iPSCs to generation of polarized macrophages. Briefly, to produce hematopoietic progenitor cells from human iPSCs, spin EBs are formed after plating ˜8000 human iPSCs per well of a round-bottom 96-well plate in a differentiation medium containing the stem cell factor (SCF), vascular endothelial growth factor (VEGF) and bone morphogenic protein 4 (BMP4). After 6 days of culture, EBs transferred to 6-well plates in media supplemented with M-CSF and IL-3. After about 1-2 weeks, the EBs produce human macrophage progenitor cells (iPSC-MPro). Next, human iPSC-MPro are transferred to new plates in serum-free media containing M-CSF and allow to mature for 5-7 days (iPSC-M0). Macrophage can then be primed in vitro to either M1 or M2 phenotypes by treating with LPS+IFN-g or IL-4+IL-13, respectively. Representative images of each differentiation step including modified Giemsa stain of human iPSC-M0, M1 and M2 are depicted (scale; 100 μm). FIG. 1C shows a flow cytometric analysis of human iPSCs (TRA-1-81 and SSEA-4) and FIG. 1D shows a hematopoietic progenitor cells (EBs; CD34, CD31, CD43 and CD45). FIG. 1E shows a cumulative number of human iPSC-MPro generated in this system demonstrates we can continuously produce human iPSC-MPro from undifferentiated human iPSCs for more than 12 weeks at a quantity of ˜1×10⁶ cells/week/well (6-well plate). FIG. 1F shows a phenotypic characterization of human iPSC-MPro over different harvests is shown.

FIGS. 2A-2G show characterization of human iPSCs-derived Macrophages. FIG. 2A shows a flow cytometric analysis demonstrate expression of typical antigenic markers CD14, CD11b, CD68, CD86, HLA-DR and SIRP-α on human iPSC-M0 and human PB-M0 indicating that human iPSC-M0 have the same phenotype to that of human PB-M0. FIG. 2B shows a flow cytometric analysis of expression of typical M1/M2 macrophage surface markers; CD80 and CD206 respectively in human iPSC and PB-derived macrophages. FIG. 2C shows an RT-qPCR for M1 (CD80, CD40) and M2-associated genes (CD206, TGM2) in human iPSC and PB-derived macrophages (M0, M1 and M2). Data represent the mean +/−SEM of n=3 technical replicates. Statistical analysis was performed using one-way ANOVA with multiple comparisons. FIG. 2D shows a phagocytosis of carboxy late-modified polystyrene-labeled latex beads (2 hours incubation) is used to demonstrate function of human iPSC and PB-derived macrophages (M0, M1 and M2) as analyzed by flow cytometry (blue filled: cells treated with 2 μm beads; red filled: untreated cell). FIG. 2E shows a representative image of fluorescent microscopy of human iPSC-M0 without and with latex beads, respectively is shown (scale; 200 μm). FIG. 2F shows a representative histograms of bead phagocytosis of human iPSC and PB-derived macrophages (M0, M1 and M2). FIG. 2G shows an MSD analysis of cytokine expression (TNF-α, IL-6, IL-10, TARC, MDC) in human iPSC-derived macrophages (M0, M1 and M2). Data represent the mean +/−SEM of n=2 biological replicates. Statistical analysis was performed using one-way ANOVA with multiple comparisons.

FIGS. 3A-3B show gene expression analysis of different human iPSC-derived macrophages. FIG. 3A shows a heatmap analysis of differentially expressed known genes of M1 and M2 macrophages in human iPSC-M1 and human iPSC-M2 and FIG. 3B shows a select list of previously reported genes involved in tissue repair and regeneration shown in human iPSC-M1 and human iPSC-M2 via RNA sequencing (N=3 biological replicates).

FIGS. 4A-4C show analysis of in vivo efficacy of human iPSC-derived macrophage populations in rag2^{-/-} gc^{-/-} mice. FIG. 4A shows a timeline of in vivo fibrosis induction and therapeutic administration of human iPSC-M1 and iPSC-M2 macrophages. 4 groups of rag2^{-/-} gc^{-/-} mice received; corn oil only (CO) (n=9), CCL₄ only (n=14), CCL₄+human iPSC-M1 (n=13) or CCL₄+human iPSC-M2 (n=13). FIG. 4B shows liver weight to body weight ratio. Ratio was significantly lower in human iPSC-M2 treated livers compared to both CCl₄ and human iPSC-M1 (p=0.02, 0.004, respectively). FIG. 4C shows mouse livers were stained for H&E, Sirius Red, aSMA, Desmin, and F4/80. Average positive area was calculated as a percentage for all mice in each group; only representative images are shown (x4 objective). D) Quantification of staining for each marker is shown below the fields (*P<0.05, ** P<0.01, *** P<0.001).

FIGS. 5A-5B show a gene and cytokine expression analysis in liver of rag2^{-/-} gc^{-/-} mice treated with iPSC-derived macrophage populations. FIG. 5A shows a quantitative RT-PCR analysis for select markers of fibrosis. A one-way analysis of variance was used to compare all samples (*P<0.05, **P<0.01, *** P<0.001). Data are shown as fold change compared with corn oil treated controls. FIG. 5B shows analysis of a panel of cytokines/chemokines on liver homogenates performed by Ampersand Biosciences. A one-way analysis of variance was used to compare all samples (*P<0.05, **P<0.01, *** P<0.001).

FIGS. 6A-6B show a gene ontogeny (Biological process) analysis of human iPSC-MPro and human iPSC-M0 shows upregulated FIG. 6A and downregulated FIG. 6B pathways (N=3 biological replicates).

FIGS. 7A-7B show a gene expression analysis of different human iPSC- and PB-derived macrophage populations. FIG. 7B shows a heatmap of differentially expressed known markers in human PB-M1 and human PB-M2 macrophages (N=3 biological replicates). FIG. 7A shows a venn diagram of differentially expressed genes in human iPSC-M0, iPSC-M1, iPSC-M2 and overlap with their human PB-derived counterparts.

FIGS. 8A-8B show a cell surface marker and gene expression analysis of different human iPSC-and PB-derived macrophage populations. FIG. 8A shows a flow cytometric analysis demonstrates expression of typical antigenic markers CD14, CD11b, CD36, CD86 and SIRP-α on human iPSC-MPro and human PB-monocyte. FIG. 8B shows a hierarchical clustering of differentially expressed genes in human iPSC-derived macrophages (iPSC-MPro, iPSC-M0, iPSC-M1, iPSC-M2) in comparison with human PB-derived macrophages (PB-monocyte, PB-M0, PB-M1, PB-M2) via RNA sequencing (N=3 biological replicates).

FIGS. 9A-9B show an analysis of liver fibrosis regression in injured rag2^{-/-} γc^{-/-} mice. FIG. 9A shows in a pilot studyrag2^{-/-} γc^{-/-} mice were given CCl₄ to induce fibrosis and then were either harvested after the last dose (n=2) or allowed to rest for 9 days (n=2). Livers were stained for H&E, Sirius Red, αSMA, and F4/80. Average positive area was calculated as a percentage for all mice in each group; only representative images are shown (x4 objective). Quantification of staining for each marker is shown below the fields (*P<0.05, ** P<0.01, *** P<0.001). FIG. 9B shows quantitative RT-PCR analysis for select markers reveals significant reduction in fibrosis after 9 days. Data are shown as relative expression against HPRT, a housekeeping gene. A one-way analysis of variance was used to compare all samples. (*P<0.05, ** P<0.01, *** P<0.001).

FIGS. 10A-10C show an analysis of homing and survival of human iPSCs-derived macrophages in mice. FIG. 9A shows 3'10⁶ PKH-26-labeled human iPSC-M0 macrophages were injected IP in uninjured rag2^{-/-} γc^{-/-} mice and their migration was tracked over 16 days (n=2 for each time point); representative images are shown (4× magnification). FIG. 9B shows mouse livers stained with human CD68 antibody. FIG. 9C shows mouse livers stained for Sirius Red and aSMA. Average positive area was calculated as a percentage for all mice in each group; only representative images are shown (x4 objective). Quantification of staining for each marker is shown to the right of fields (*P<0.05).

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The invention provides therapeutic cellular composition, methods of use and methods of deriving macrophages from human induced pluripotent stem cells (iPSCs) and differentiating them to M1 and/or M2 phenotypes. The disclosure demonstrates the efficiency of human iPSC-derived macrophages to ameliorate liver fibrosis in an established immunodeficient Rag2^{-/-} γc^{-/-} mouse model of liver fibrosis. Although Rag2^{-/-} γc^{-/-} mice lack T and B cells, they retain the myeloid cell population, and develop significant liver fibrosis in response to CCI₄-induced toxic liver injury. Administration of human iPSC-derived macrophages improved liver function, reduced inflammation and development of liver fibrosis, and improved hepatocyte function. Human iPSCs are able to generate sufficient numbers of human macrophages with an anti-inflammatory phenotype to provide a storable, off-the-shelf cell-based therapy for liver diseases.

The invention provides therapeutic cellular compositions and methods to generate macrophages from human induced pluripotent stem cells (iPSCs) and differentiate them to the M1 phenotype and/or the M2 phenotype.

In embodiments, the invention provides methods to treat damaged liver tissue, comprising administering to a subject in need thereof an effective amount of a composition comprising a plurality of macrophages derived from human induced pluripotent stem cells (iPSCs), wherein the macrophages are polarized to a pro-inflammatory M1 phenotype and/or an anti-inflammatory M2 phenotype. In embodiments, the subject has liver fibrosis. In embodiments, the administration reduces fibrogenic gene expression and liver disease associated histological markers. In embodiments, the invention contemplates administering one or both M1 and M2 phenotypes.

In embodiments, the invention provides compositions comprising a plurality of macrophages derived from human induced pluripotent stem cells (iPSCs), wherein the macrophages are polarized to a pro-inflammatory M1 phenotype and/or an anti-inflammatory M2 phenotype. In embodiments, the M1 macrophages express elevated CD80, TNF-α and IL-6. In embodiments, the M2 macrophages express elevated CD206, CCL17, and CCL22.

In embodiments, the invention provides methods for the manufacture of a cellular composition comprising deriving a plurality of macrophages from human induced pluripotent stem cells (iPSCs), and polarizing the macrophages to a pro-inflammatory M1 phenotype and an anti-inflammatory M2 phenotype, thereby manufacturing the cellular composition. In embodiments, the macrophages are polarized to pro-inflammatory M1 in presence of an effective amount of LPS+IFN-γ. In embodiments, the M1 macrophages express elevated CD80, TNF-α and IL-6. In embodiments, the macrophages are polarized to anti-inflammatory M2 phenotypes in presence of an effective amount of IL-4+IL-13. In embodiments, the M2 macrophages express elevated CD206, CCL17, and CCL22.

In embodiments, the invention provides a pharmaceutically acceptable composition comprising a plurality of macrophages derived from human induced pluripotent stem cells (iPSCs), wherein the macrophages are polarized to a pro-inflammatory M1 phenotype and/or an anti-inflammatory M2 phenotype, as described herein.

The practice of the present invention may employ conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook et al, 1989) Cold Spring Harbor Press; Oligonucleotide Synthesis (M J. Gait, ed., 1984); Methods in Molecular Biology, Humana Press; Cell Biology: A Laboratory Notebook (J .E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C C. Blackwell, eds.); Gene Transfer Vectors for Mammalian Cells (J. M. Miller and M. P. Calos, eds., 1987); Current Protocols in Molecular Biology (F. M. Ausubel et al , eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (CA. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al, eds., J. B. Lippincott Company, 1993). Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises,” “comprising,” “includes,” “including,” “has,” “having,” “contains”, “containing.” “characterized by,” or any other variation thereof, are intended to encompass a non-exclusive inclusion, subject to any limitation explicitly indicated otherwise, of the recited components. For example, a cell, a pharmaceutical composition, and/or a method that “comprises” a list of elements (e.g., components, features, or steps) is not necessarily limited to only those elements (or components or steps), but may include other elements (or components or steps) not expressly listed or inherent to the cell, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10%of the recited value, within 5%of the recited value, or within 2%of the recited value.

As used herein, and unless otherwise specified, the term “subject” or “patient” is defined herein to include animals such as mammals, including, but not limited to, primates (e.g., humans), cows, sheep, goats, horses, dogs, cats, rabbits, rats, mice, and the like. In specific embodiments, the subject is a human. The terms “subject” and “patient” are used interchangeably herein in reference, for example, to a mammalian subject, such as a human.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soy bean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

As used herein, “induced pluripotent stem cell” or “iPSC cell” or “iPSCs” are used to refer to cells, derived from somatic cells, that have been reprogrammed back to an pluripotent state that are capable of proliferation, selectable differentiation, and maturation.

As used herein, a composition containing a “purified cell population” or “purified cell composition” means that at least 30%, 50%, 60%, typically at least 70%, and more preferably 80%, 90%, 95%, 98%, 99%, or more of the cells in the composition are of the identified type.

Generally, techniques for differentiating an induced pluripotent cell involve modulation of specific cellular pathways, either directly or indirectly, using polynucleotide-, polypeptide- and/or small molecule-based approaches. The developmental potency of a cell may be modulated, for example, by contacting a cell with one or more modulators. “Contacting”, as used herein, can involve culturing cells in the presence of one or more factors (such as, for example, small molecules, proteins, peptides, etc.). In some embodiments, a cell is contacted with one or more agents to induce cell differentiation. Such contact, may occur for example, by introducing the one or more agents to the cell during in vitro culture. Thus, contact may occur by introducing the one or more agents to the cell in a nutrient cell culture medium. The cell may be maintained in the culture medium comprising one or more agents for a period sufficient for the cell to achieve the differentiation phenotype that is desired.

Differentiation of stem cells requires a change in the culture system, such as changing the stimuli agents in the culture medium or the physical state of the cells. A conventional strategy utilizes the formation of embryoid bodies (EBs) as a common and critical intermediate to initiate the lineage-specific differentiation. EBs are three-dimensional clusters that have been shown to mimic embryo development as they give rise to numerous lineages within their three-dimensional area. Through the differentiation process simple EBs (for example, aggregated pluripotent stem cells elicited to differentiate) continue maturation and develop into a cystic EB at which time, they are further processed to continue differentiation. EB formation is initiated by bringing pluripotent stem cells into close proximity with one another in three-dimensional multilayered clusters of cells. Typically, this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. This may be followed by additional stimulation differentiating the iPSCs.

Illustrative methods for making and using engineered cells are provided in Int'l Pat. Appl. Nos. WO 2013/163171 A1, WO 2017/078807 A1, and WO 2018/147801 the disclosures of which are incorporated by reference herein in their entireties.

As used herein, “differentiate” or “differentiated” are used to refer to the process and conditions by which immature (unspecialized) cells acquire characteristics becoming mature (specialized) cells thereby acquiring particular form and function. Stem cells (unspecialized) are often exposed to varying conditions (e.g., growth factors and morphogenic factors) to induce specified lineage commitment, or differentiation, of said stem cells. The process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

In some embodiments, one or more of the media of the culture platform is a feeder-free environment, and optionally is substantially free of cytokines and/or growth factors. In some embodiments, the cell culture media contains supplements such as serums, extracts, growth factors, hormones, cytokines and the like. Generally, the culture platform comprises one or more of stage specific feeder-free, serum-free media, each of which further comprises one or more of the followings: nutrients/extracts, growth factors, hormones, cytokines and medium additives. Suitable nutrients/extracts may include, for example, DMEM/F-12 (Dulbecco's Modified Eagle Medium/Nutrient Mixture F-12), which is a widely used basal medium for supporting the growth of many different mammalian cells; KOSR (knockout serum replacement); L-glut; NEAA (Non-Essential Amino Acids). Other medium additives may include, but not limited to, MTG, ITS, (ME, anti-oxidants (for example, ascorbic acid). In some embodiments, a culture medium of the present invention comprises one or more of the following cytokines or growth factors: epidermal growth factor (EGF), acidic fibroblast growth factor (aFGF), basic fibroblast growth factor (bFGF), leukemia inhibitory factor (LIF), hepatocyte growth factor (HGF), insulin-like growth factor 1 (IGF-1), insulin-like growth factor 2 (IGF-2), keratinocyte growth factor (KGF), nerve growth factor (NGF), platelet-derived growth factor (PDGF), transforming growth factor beta (TGF-B), bone morphogenetic protein (BMP4), vascular endothelial cell growth factor (VEGF) transferrin, various interleukins (such as IL-1 through IL-18), various colony-stimulating factors (such as granulocyte/macrophage colony-stimulating factor (GM-CSF)), various interferons (such as IFN-γ) and other cytokines having effects upon stem cells such as stem cell factor (SCF) and erythropoietin (EPO). These cytokines may be obtained commercially, for example from R&D Systems (Minneapolis, Minn.), and may be either natural or recombinant. In some other embodiments, the culture medium of the present invention comprises one or more of bone morphogenetic protein (BMP4), insulin-like growth factor-1 (IGF-1), basic fibroblast growth factor (bFGF), vascular endothelial growth factor (VEGF), hematopoietic growth factor (for example, SCF, GMCSF, GCSF, EPO, IL3, TPO, EPO), Fms-Related Tyrosine Kinase 3 Ligand (Flt3L); and one or more cytokines from Leukemia inhibitory factor (LIF), IL3, IL6, IL7, IL11, IL15. In some embodiments, the growth factors/mitogens and cytokines are stage and/or cell type specific in concentrations that are determined empirically or as guided by the established cytokine art.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate,” or “maintain,” refers to the sustaining, propagating (growing) and/or differentiating of cells outside of tissue or the body, for example in a sterile plastic (or coated plastic) cell culture dish or flask. “Cultivation,” or “maintaining,” may utilize a culture medium as a source of nutrients, hormones and/or other factors helpful to propagate and/or sustain the cells.

Multipotent hematopoietic stem cells provide the basis of two major progenitor cell lineages. The first cell lineage is the common lymphoid progenitor cell lineage, wherein a multipotent hematopoietic stem cell (hemocytoblast) differentiates into a lymphoid progenitor cell, which has the capability to further differentiate into a natural killer cell, T lymphocyte, or B lymphocyte; or differentiate even further from a B lymphocyte to a plasma cell. The other major cell lineage is the common myeloid progenitor cell lineage, wherein a hemocytoblast differentiates into a myeloid progenitor cell, which has the capability to further differentiate into a megakaryocyte, erythrocyte, platelet, mast cell, or myeloblast; or differentiate even further from a myeloblast to a basophil, neutrophil, eosinophil, or monocyte; or yet further differentiate from a monocyte to a macrophage.

### EXAMPLES

### Efficient Generation of Human iPSC-Derived Macrophages

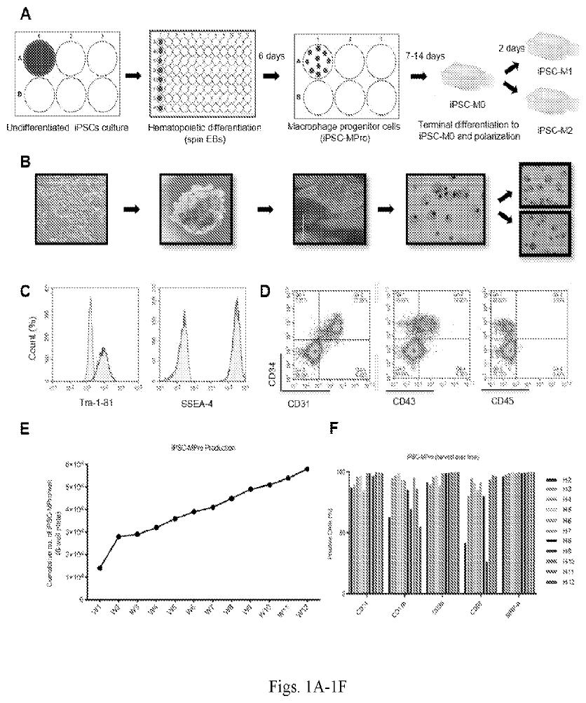

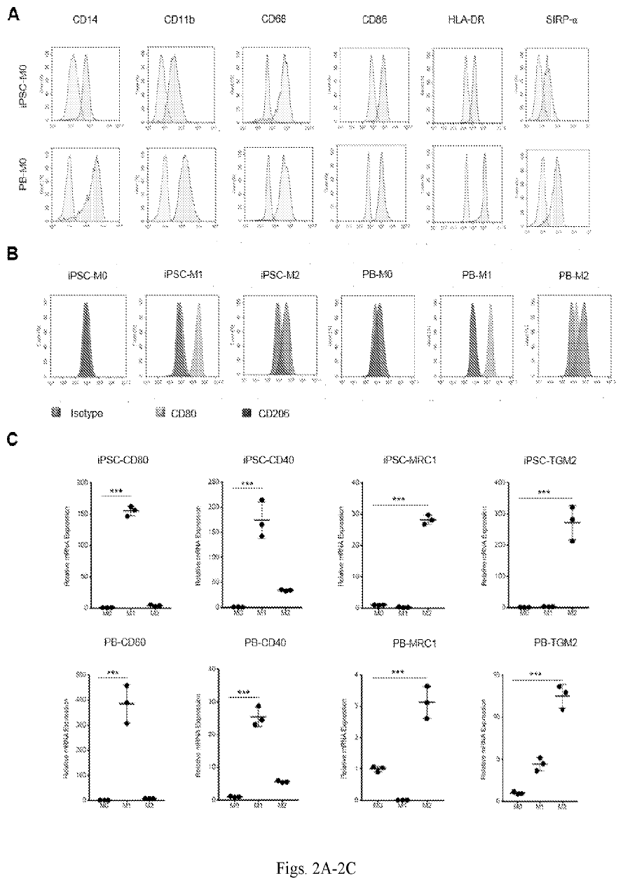

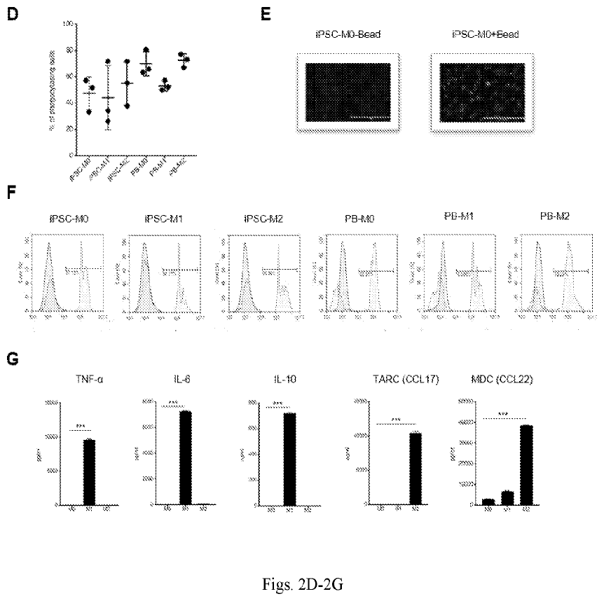

This example demonstrates a novel method to derive hematopoietic progenitor cells from human iPSCs for efficient production of human macrophages. A schematic representation of the macrophage production pipeline is depicted in FIGS. 1A and 1B. Briefly, human iPSCs expressing typical pluripotency markers (TRA-1-81⁺ and SSEA-4⁺; FIG. 1C) were aggregated in defined serum-free media supplemented with SCF, VEGF, BMP4 and Rock-Y to produce embryoid bodies (EBs), also considered hematopoietic organoids. After 6 days, the EBs in these conditions begin to generate hematopoietic and endothelial cells (CD34⁺, CD31⁺, CD43⁺; FIG. 1D) 15. The EBs were then transferred into flat bottom plates in macrophage differentiation media I containing M-CSF and IL-3. Under these conditions, EBs generate human macrophage progenitor cells (iPSC-MPro) that can then be supported to terminal differentiation towards human iPSC-M0 in macrophage differentiation media II containing M-CSF for 7 days. Human iPSC-M0 can then be polarized to human iPSC-M1 or human iPSC-M2 phenotypes in presence of LPS+IFN-γ or IL-4+IL-13, respectively as described in detail below.

The EBs were able to continuously produce human iPSC-MPro for more than 12 weeks at a quantity of 5×10⁵ to 1×10⁶ cells/week/well in 6-well plate format (FIG. 1E). These cells can be routinely harvested from suspension and further differentiated to human iPSC-M0 and subsequent human iPSC-M1 or human iPSC-M2 populations. Phenotypic characterization of human iPSC-MPro showed uniform and consistent expression of typical macrophage cell surface markers (CD14, CD11b, CD36, CD68 and SIRP-α) (FIG. 1F).

### Human iPSC-Derived Macrophage Populations Show Similar Phenotypic Characteristics to Human Peripheral Blood-Derived Macrophages

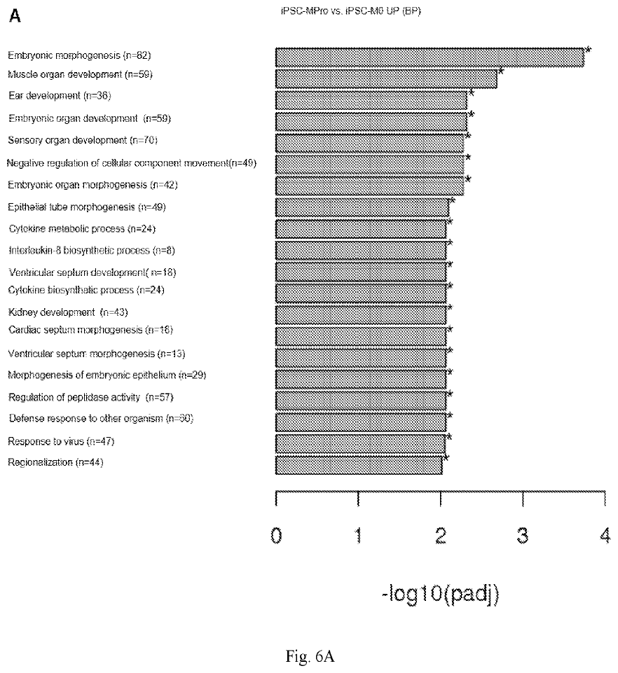

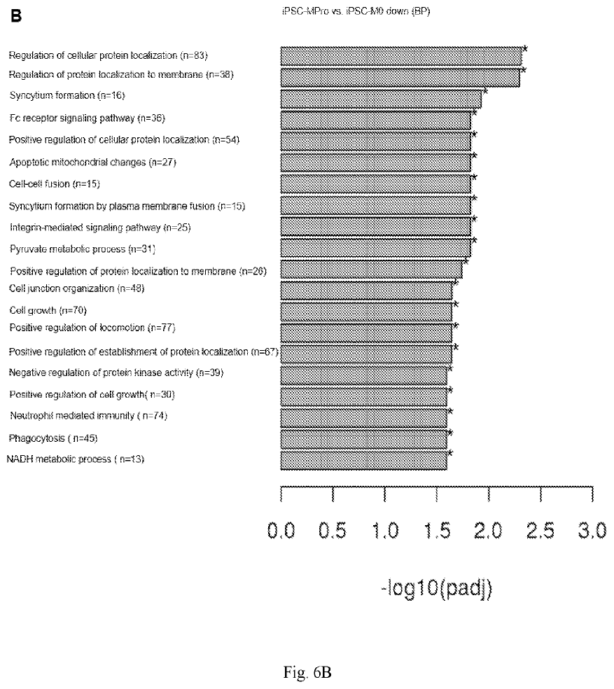

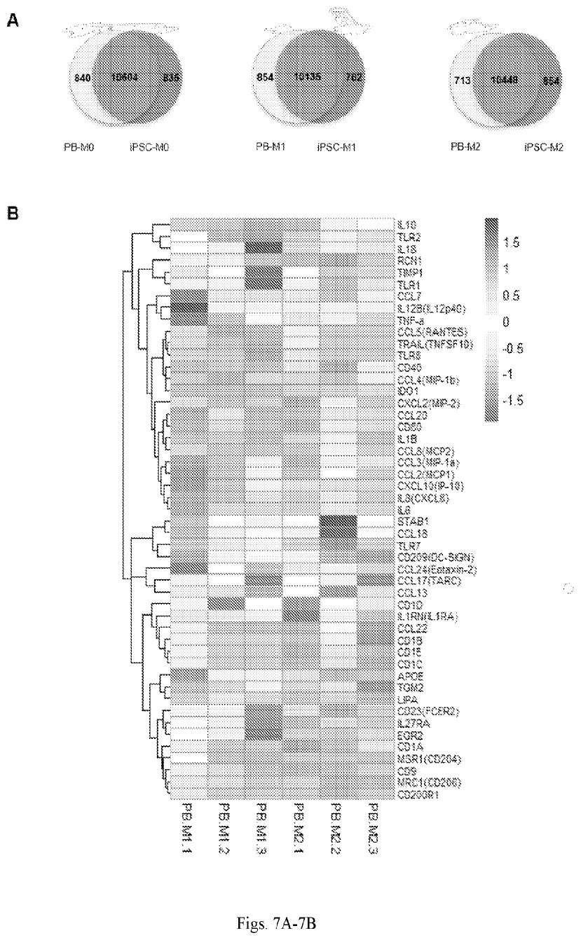

To test whether human iPSC-MPro can be differentiated to human iPSC-M0 similar to human PB-M0, human iPSC-MPro and human PB-monocytes were terminally differentiated for 7 days in differentiation medium II and expression profile of a panel of typical macrophage cell surface markers were analyzed in human iPSC-M0 and human PB-M0 respectively (FIG. 2A). The LPS receptor CD14, CD11b or Macrophage-1 antigen (Mac-1), pan-macrophage marker CD68, costimulatory molecule CD86 (B7-2), MHC class II (HLA-DR) and signal regulatory protein a (SIRPα) which interacts with a broadly expressed transmembrane protein CD47, were all expressed at high levels in human iPSC-M0 and human PB-M0. Although phenotypic markers of human iPSC-MPro did not change upon differentiation to human iPSC-M0, gene ontogeny analysis revealed that some biological processes such as Fc receptor signaling, and phagocytosis were significantly up-regulated after differentiation to M0 while pathways involved in morphogenesis and development were enhanced in human iPSC-MPro population (FIGS. 6A and 6B).

To assess whether human iPSC-derived macrophages resemble human PB-derived macrophages upon activation, cells were polarized to either M1 (in presence of IFN-γ and LPS) or M2 (in presence of IL-4 and IL-13) macrophages. Phenotype, gene expression, phagocytic activity and their ability to secrete cytokine/chemokine were evaluated. Flow cytometric analysis demonstrated that human iPSC-M1 and human PB-M1 exhibited similar expression of CD80; a classical M1 marker while M2 polarization resulted in more pronounced expression of mannose receptor CD206 (MRC1) in human iPSC-M2 compared to human PB-M2 (FIG. 2B).

RT-qPCR analysis of M1 and M2-associated genes showed that pro-inflammatory phenotype markers CD80 and CD40 were upregulated in both human iPSC-M1 and human PB-M1. M2 culture conditions lead to increased expression of MRC1 and transglutaminase 2 (TGM2), with this expression notably higher in iPSC-M2 compared to PB-M2 (FIG. 2C).

### Human iPSC-Derived Macrophage Populations Show Similar Functional Characteristics to Human Peripheral Blood-Derived Macrophages

Quantification of phagocytic capacity of 2 μm latex beads by different human iPSC and PB-derived macrophage populations demonstrated that while human iPSC-M1 and human PB-M1 had slightly lower phagocytic activity, human iPSC-M2 and human PB-M2 showed almost similar levels of phagocytosis compared to their M0 counterparts (FIG. 2D-F). Overall, bead phagocytosis analysis demonstrates effective phagocytosis in all human iPSC and PB-derived macrophage populations.

Cytokine expression profile of human iPSC-derived macrophages was evaluated by MSD assay (FIG. 2G). TNF-α and IL-6; two key pro-inflammatory cytokines together with IL-10 had remarkably high expression in the supernatants of human iPSC-M1 and only background level was detected in the supernatant of either human iPSC-M0 or human iPSC-M2. Corresponding upregulation of CCL17 (TARC) and CCL22 (MDC) was demonstrated in M2 activated macrophages. Taken together, the results demonstrate that human iPSC-derived macrophages share phenotypical and functional characterization to that of human PB-derived macrophages.

### RNA Expression Profile of Human iPSC-M1 and Human iPSC-M2 Macrophage Sub-Types Demonstrate Distinct Pro-Inflammatory and Anti-Inflammatory Phenotypes

Detailed characterization of more differentiated human iPSC-derived macrophage sub-types were assessed in RNA-seq analysis and compared with human PB counterparts. There were three aims: first, to assess the expression of known M1-and M2-specific markers in human iPSC and human PB-derived macrophages: second, to determine the expression of genes that are involved in liver fibrosis repair and regeneration: and third, to compare gene expression profile of human iPSC and human PB-derived macrophages.

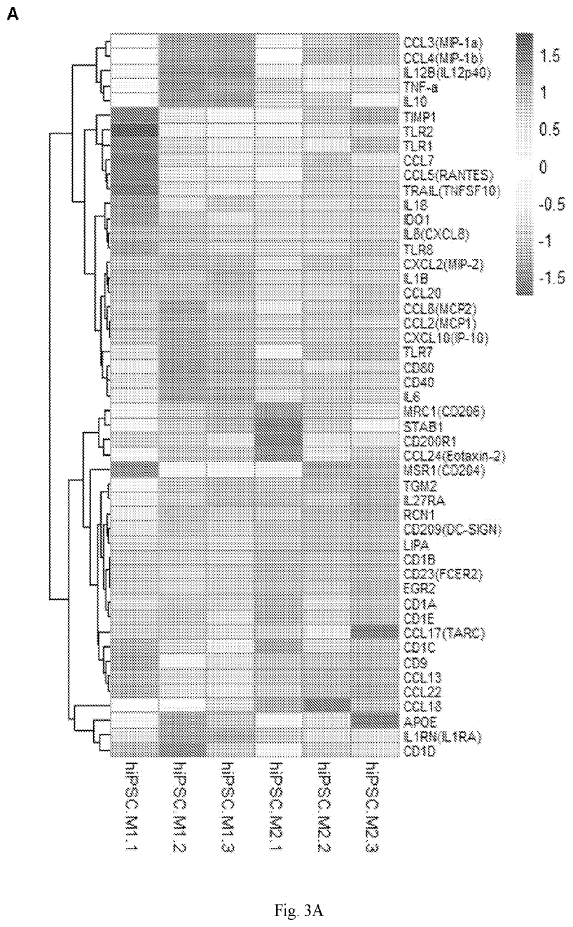

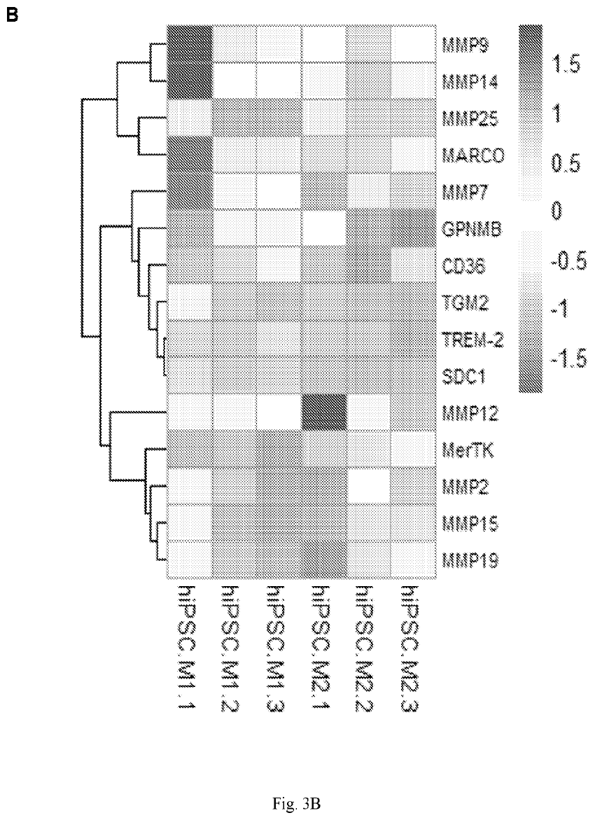

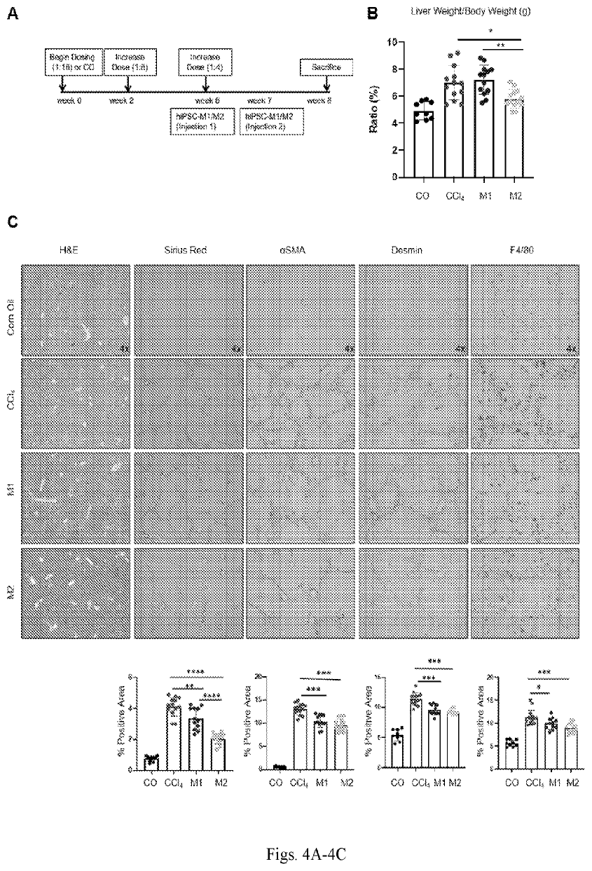

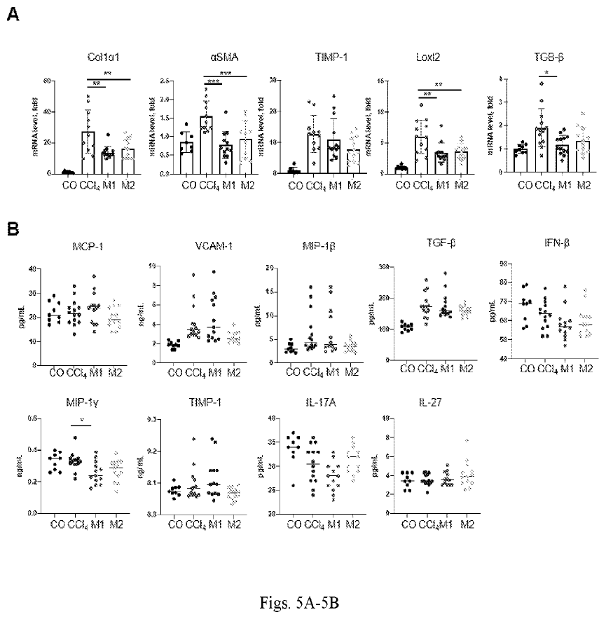

Consistent with the RT-qPCR and MSD data, heatmap analysis of M1- and M2-associated genes demonstrated distinct expression profiles. CD80, CD40, TNF-α, IL-6 were upregulated in M1 and likewise CD206, TGM-2, CCL17 and CCL22 in M2 macrophages. Genes associated with inflammatory responses such as toll-like receptors (TLRs 1, 2, 7, 8) and inflammatory cytokines/chemokines [TNF-γ, IL-6, IL-1b, IL-12, CCL2, CCL3 and TRAIL (TNFSF10)] were significantly upregulated in human iPSC-M1 compared to human iPSC-M2 macrophages. In contrast, anti-inflammatory markers such as CD9, IL-IRN, IL-27R, RCN1 ¹⁶⁻¹⁹ were only present in human iPSC-M2 subtype. Moreover, detailed characterization of human iPSC-M2 versus human iPSC-M1 sub-type, demonstrated elevated levels of scavenger receptors CD204 (SR-AI), CD206 (MRC1), CD209 (DC-SIGN) and Stabilin-1 (STAB-1)²⁰ in human iPSC-M2 (FIG. 3A). The same pattern was also observed in human PB-M1 and human PB-M2 subtypes (FIG. 7A). Interestingly, expression of several critical genes that are related to phagocytosis were differentially expressed in human iPSC-M1 and human iPSC-M2 sub-types. Specifically, MerTK, CD36, TREM-2 had higher expression in human iPSC-M2 while MARCO increased in human iPSC-M1. Similar pattern was observed for MMPs, while both groups shared similar expression of MMP-9 and 12, MMP-25 and MMP-7 were solely detected in human iPSC-M1 and human iPSC-M2, respectively (FIG. 3B). Additionally, genes involved in tissue regeneration such as melanoma-associated transmembrane glycoprotein (Gpnmb)²¹, Syndecan 1 (SDC-1)^{22,23} were highly upregulated in human iPSC-M2 subtype. These data suggest that although both human iPSC-M1 and human iPSC-M2 subtypes share expression of some important genes involved in tissue repair and regeneration, human iPSC-M2 demonstrated more prominent expression of the related genes.

Moreover, gene ontogeny analysis of human iPSC and PB-derived macrophage populations demonstrated that human iPSC and human PB-derived M0, M1 and M2 largely shared transcriptomic profiles (86% between corresponding sub-types) while also revealing lineage-specific differentially expressed genes (FIG. 7B).

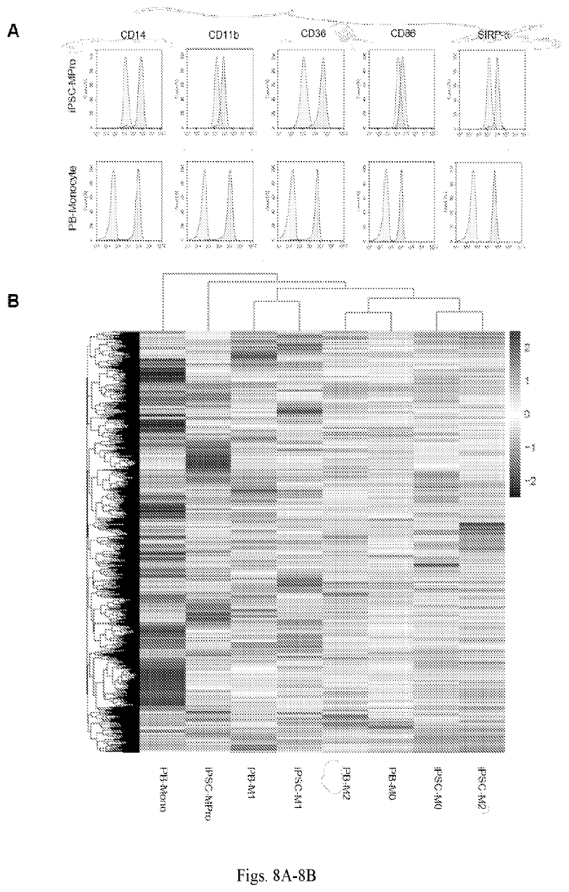

Although human iPSC-MPro were originally thought to be equivalent to human monocytes and show similar phenotypic expression to PB-monocyte (FIG. 8A), gene expression analysis supports that these two populations belong to a different cell ontogeny (FIG. 8B). Indeed, unlike human PB-monocytes which arise from hematopoietic stem cells, these results support studies that suggest human iPSC-MPro originate from yolk sac hematopoietic progenitor cells, self-renew during adult life through a Myb-independent proliferation mechanism (independent from HSCs) and are more similar to tissue resident macrophages^{12,24}. In addition, hierarchical heatmap clustering of differentially expressed genes revealed close proximity of human iPSC-M2 and human iPSC-M0 and likewise in human PB-M2 and human PB-M0 macrophages (FIG. 8B), similar to other studies of ESC and iPSC-derived macrophages^{12,25}.

### CCL-Injured Mice Rag2^{-/-}γc^{-/-} Mice Develop Liver Fibrosis

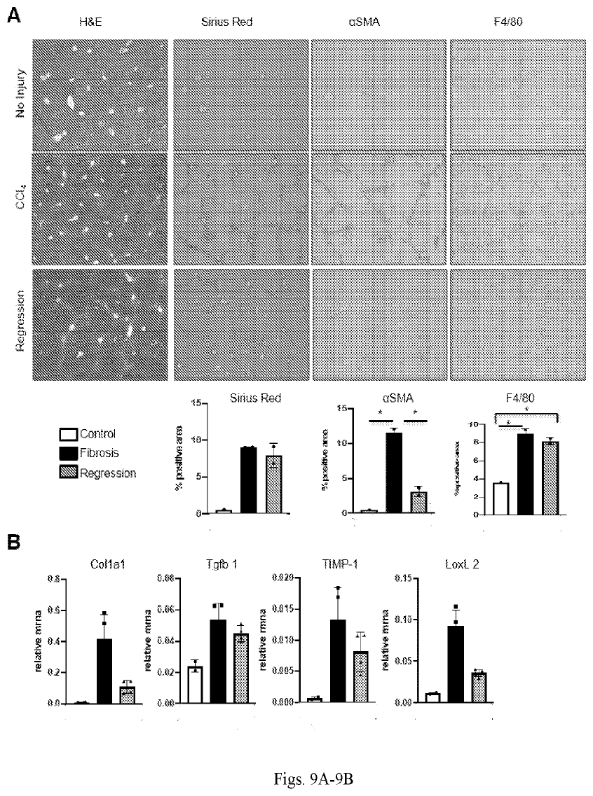

In a pilot study to evaluate liver fibrosis in an immunodeficient Rag2^{-/-}γc^{-/-} mice, fibrosis was induced by gradual increase of CCl₄ administration throughout the period of 8 weeks (CCl₄ in corn oil, twice a week via oral gavage). In comparison with the control mice that received corn oil alone, CCl₄-injured Rag2^{-/-}γc^{-/-} mice developed bridging fibrosis, shown by increased area of Sirius Red staining (4-fold), and activation of aSMA⁺ and Desmin⁺ aHSCs/myofibroblasts (FIG. 9A). Development of liver fibrosis in CCl₄-injured Rag2^{-/-}γc^{-/-} mice was associated with the increased recruitment and activation of F4/80⁺ macrophages, and increased expression of the fibrogenic genes Colla1, aSMA, TIMP1, and LoxL2 in total tissue mRNA (FIG. 9B).

Evaluation of fibrosis regression (9 days after cessation of CCl₄) showed suppression in inflammatory responses and downregulation of ECM producing myofibroblasts, shown by decreased Sirius Red and aSMA histology, as well as reduced expression of fibrogenic genes (FIGS. 9A-9B). Based upon this data, a therapeutic regimen of iPSC administration was adopted for follow up experiments to recapitulate treatment of patients with liver fibrosis where the etiological agent remains present.

### Administration of Human iPSC-Derived M2 Macrophages Ameliorates Liver Fibrosis in Rag2^{-/-}γc^{-/-} Mice

To determine the therapeutic effect of human iPSC-derived macrophages on liver fibrosis in CCl₄-injured Rag2^{-/-}γc^{-/-} mice, human iPSC-M1 or-M2 (5×10⁶ cells/mouse, 2× IP injection) were administered during the CCl4 regimen, with a 1-week interval between doses (FIG. 4A). Control mice were given corn oil only. Mice were sacrificed one week after the second and final iPSC-M1 or-M2 injection. Liver tissue and serum were collected and analyzed (FIG. 4A-C). The liver weight to body weight ratio was significantly lower in human iPSC-M2 treated Rag2^{-/-}γc^{-/-} mice compared to both CCl4-injured or CCl4-injured, iPSC-M1 treated mice (p<0.01) (FIG. 4B) and was associated with the improvement of liver gross morphology (not shown). Quantification of Sirius Red staining revealed a significant decrease of collagen deposition in CCl₄-injured Rag2^{-/-}γc^{-/-} mice administered with human iPSC-M2 (2.0% total positive area) compared to CCl4 treatment alone (4.1%, p<0.001) as well as compared to iPSC-M1 treated mice (3.4%, p<0.001). Interestingly, mice treated with human iPSC-M1 also showed a significant reduction in fibrosis compared to CCl₄ treated mice, despite the pro-inflammatory profile of these cells ex-vivo (p=0.003).

Expression of aSMA was significantly decreased in Rag2^{-/-}γc^{-/-} mice administered with human iPSC-M2 (9.5%) treated groups compared to CCl₄ treatment alone (13.0%, p<0.0001) (FIG. 4C). Hepatic stellate cells (HSCs) are known to activate and deposit extracellular matrix proteins in response to injury²⁶. In conjunction, staining for desmin⁺ activated HSCs was significantly reduced in human iPSC-M1 (9.6%) and -M2 (9.3%) treated groups compared to CCl4 treatment alone (11.4%) (p<0.0001). Staining for F4/80⁺ inflammatory macrophages was significantly reduced in human iPSC-M1 (9.9%) and human iPSC-M2 (9.0%) treated groups compared to CCl₄ treatment alone (11.3%) (p=0.03, 0.0002, respectively) (FIG. 4C). Quantification of staining for histology is shown (FIG. 4D).

Together, these data revealed that therapeutic administration of both human iPSC-M1 and-M2 cells in vivo results in reduced fibrosis and inflammation. While iPSC-M2 demonstrated stronger downregulation of clinically relevant fibrotic markers, it was unexpectedly also observed profound antifibrogenic potential in iPSC-M1. Resolution of fibrosis in the presence of iPSC-M1 may be indicative of a shift in phenotype upon injection to the peritoneal cavity of Rag2^{-/-}γc^{-/-} mice, though the mechanism of this shift is unknown. Ma et al.²⁷report on the therapeutic potential of M1 polarized BMDMs to ameliorate fibrosis via the recruitment of endogenous macrophages and NK cells that promote HSC apoptosis through MMPs and TRAIL²⁷. While Rag2^{-/-}γc^{-/-} mice lack NK cells, iPSC-M1 cells can potentially recruit MMP secreting-endogenous macrophages with powerful antifibrotic effects during injury.

### Gene Expression Profiles are Less Inflammatory in Human Ipsc-Derived Macrophage-Treated Mice.

Expression of fibrogenic markers was evaluated by RT-qPCR (FIG. 5A). Collagenlal (Collal) and aSMA, ECM protein components of fibrotic scars deposited by activated HSCs during injury to the liver, were significantly reduced in iPSC-M1 and -M2 treated mice compared to CCl₄ only (p=0.001, 0.008 for Collal in iPSC-M1 and -M2, respectively; p=0.0003, 0.007 for αSMA iPSC-M1 and-M2, respectively). LoxL2, a fibrosis-associated enzyme responsible for crosslinking connective tissues in scar formation, was significantly lower in both iPSC-M1 and-M2 treated mice (p=0.004, 0.009). TGF-ß1 was also significantly downregulated in both iPSC-M1 and -2 treated mice (p=0.01). TIMP-1 showed a non-significant decrease in the liver of fibrotic mice after both iPSC-M1 or-M2 administration, with markedly lower levels in the human iPSC-M2 group. Notably, there was no significant differences in gene expression profiles between iPSC-M1 and -M2 treated animals. These data support of a supposed shift, or loss of phenotype, for pro-inflammatory iPSC-M1 cells upon introduction to the peritoneal cavity.

### Cytokine Profile in Livers of Mice Treated With Human iPSC-Derived Macrophages is Less Fibrogenic/Inflammatory

Cytokine expression profile of total tissue homogenate was analyzed by luminex assay (FIG. 5B). Analysis of several pro-inflammatory cytokines/chemokines including MCP1, VCAM-1 and MIP-1β showed a reduced non-significant trend in human iPSC-M2 treated animals compared to CCl₄ treatment alone. While the level of TGF-β and IFN-β in liver decreased slightly in both human iPSC-M1 and iPSC-M2 groups, MIP-1γ only showed a significant decrease in iPSC-M1 group. Consistent with the trend we observed in total tissue gene expression analysis, TIMP-1 expression decreased more noticeably in the iPSC-M2 treated group compared to CCl₄ treatment alone. Levels of IL-17A and IL-27, two cytokines that are reported to have a collaborative role in liver regeneration 28 were elevated in liver of mice treated with human iPSC-M2 compared to CCl₄ treatment alone.

Interestingly, in corroboration with other in vivo findings, there were no significant differences between cytokine profiles in iPSC-M1 and -M2 treated livers. This similarity is surprising due to the dramatic, classic pro-inflammatory profile of human iPSC-M1 and anti-inflammatory profile of human iPSC-M2 ex vivo (FIG. 3). Because this data shows that the macrophages were indeed polarized to M1 and M2 phenotypes, it was expected that fibrosis would be exacerbated by iPSC-M1. Conversely, iPSC-M1 and -M2 had a comparable therapeutic effect on fibrosis and followed the same trends in cytokine expression, suggesting that M1 likely undergo a shift to an anti-inflammatory phenotype when injected into mice.

Overall, these data support the therapeutic administration of human iPSC-derived macrophages, and in particular iPSC-M2 macrophages, to ameliorate liver fibrosis by decreasing pro-inflammatory cytokines in the liver microenvironment, thus improving liver function and regeneration.

### Human iPSC-Derived Macrophages do not Mainly Localize Into Liver Tissue After Intraperitoneal Administration

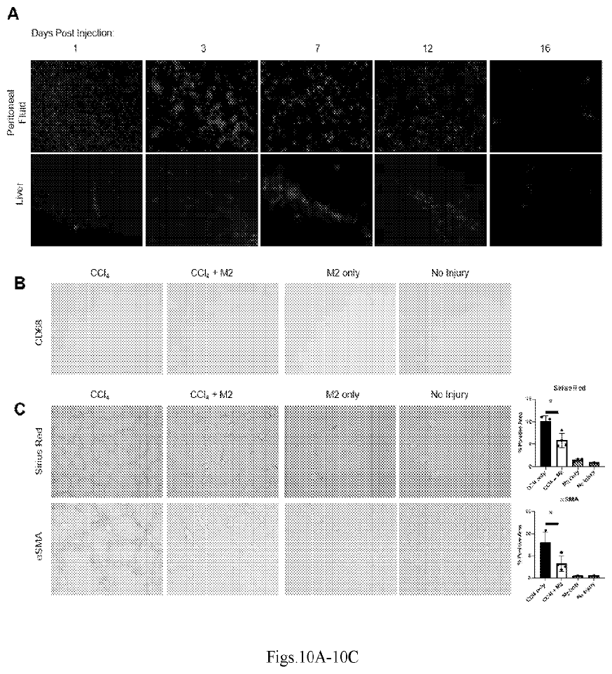

In a pilot study, 3×10⁶ PKH-26-labeled human iPSC-M0 macrophages were injected IP in uninjured mice and tracked over 16 days using UV microscopy. Cells remained primarily in the peritoneal cavity and only few cells were detected in the capsules of the liver (FIG. 10A), spleen, pancreas, and kidneys (not shown).

In a follow up study, 20×10⁶ TdTomato-expressing human iPSC-M2 macrophages were injected IP during the course of CCl₄ induced injury. The goals were to track if macrophages migrate to the liver, and to evaluate whether a larger dose of human iPSC-M2 macrophages would have a more profound impact on fibrosis reduction. Staining with human CD68 antigen (a marker of myeloid cells) on liver tissue showed that human iPSC macrophages could not be detected after IP administration into either uninjured or CCl4-injured mice treated with 1 dose of 20×10⁶ human iPSC-M2 macrophages (FIG. 10B). Although homing to the liver by iPSC-M2 macrophages was observed, they produced a robust therapeutic effect on liver injury. Cells introduced via IP injection may transiently pass through the liver resulting in changes in the microenvironment and production of anti-inflammatory factors with more persistent impacts. Alternatively, injected iPSC macrophages may secrete cytokines directly into the peritoneal cavity, leading to absorption of these anti-inflammatory factors by peritoneal vasculature into portal tract²⁹.

Consistent with previous findings, administration of an increased dose of human iPSC-M2 macrophages (20×10⁶), resulted in significant reduction in fibrosis (FIG. 10C). Collagen deposition, shown by Sirius Red staining, was significantly reduced in human iPSC-M2 macrophage injected mice compared with CCl₄ treated mice (p=0.01). aSMA staining was also significantly reduced in mice treated with 20×106 human iPSC-M2 macrophages compared with mice given CCl₄ only (p=0.02). Notably, injection of 20×106 human iPSC-M2 macrophages into either uninjured or CCl₄-injured mice did not result in any notable toxicity, suggesting that administration of human iPSC-derived macrophages is safe and tolerable. These data confirmed the initial results that administration of human iPSC-derived macrophages, particularly iPSC-M2 polarized cells, have a significant impact on ameliorating fibrosis, further highlighting the potential of human iPSC-derived macrophages as an anti-fibrotic liver therapy.

The present study provides evidence that human iPSC-derived macrophages, especially human iPSC-M2, improve liver fibrosis and stimulate regeneration in CCl4-injured mice. This study is believed to be the first to evaluate the administration of human iPSC-derived macrophages in an immunodeficient mouse model of liver injury, although other studies have investigated the effect of mouse BM-and ESC-derived macrophages^{4,12}. Moreover, the mouse BM-M2 population has also been shown to play a crucial reparative role in cardiac infarction³⁰.

This example utilized an efficient method using fully defined feeder-and serum-free differentiation protocol for rapid generation of large numbers of human iPSC-derived macrophages which exhibited characteristic macrophage morphology and expressed macrophage specific cell surface antigens. This methodology provides a critical step towards obtaining clinical scale production of human iPSC-derived macrophages for cell therapy approaches, including liver fibrosis treatment. Macrophages play an important role in regulating inflammatory responses, so we tested these human iPSC-M1 and M2 polarized macrophages in a mouse model of CCl₄-induced liver fibrosis. Although the data demonstrates that both human iPSC-macrophage populations including iPSC-M1 and iPSC-M2 resulted in fibrosis attenuation/regression in the CCl₄ model of liver fibrosis, administration of human iPSC-M2 was overall more promising. In both groups, clinical markers of fibrosis were significantly reduced as indicated by staining of Sirius Red, aSMA and desmin. Of note, the clinically relevant fibrosis marker Sirius Red, a marker of collagen deposition, demonstrated a more significant and remarkable reduction after iPSC-M2 injection.

Several potential mechanisms mediate fibrosis regression and contribute to the regenerative responses including the activation of collagenase- and MMP-secreting macrophages in parallel with decreased activity of TIMPs³¹⁻³⁴. Notably, significant expression of MMP-2, 7, 15 and 19, and reduced levels of TIMP-1 was observed in human iPSC-M2 macrophages, which could explain why collagen deposition was more significantly reduced in iPSC-M2-treated mice compared to iPSC-M1. Macrophages also act as professional phagocytes with the capacity to engulf cellular debris and apoptotic cells, and they play a critical role in the resorption of fibrous scar 35,36. The human iPSC-M2 macrophages expressed high levels of surface receptors MerTK, CD36, TREM-2 which have previously reported to promote tissue repair³⁷⁻³⁹, as well as genes that are which have previously reported to promote tissue repair involved in tissue regeneration such as Gpnmb 21 and SDC-1^{22,23}.

However, a strong therapeutic effect was observed in human iPSC-M1 injected mice, which was attributed to expression of several members of the MMP family, phagocytosis receptor MARCO, and anti-inflammatory cytokine IL-10 (FIG. 2G). While the anti-inflammatory cytokine IL-10 was expected to be upregulated upon M2 activation, high levels of IL-10 in M1 supernatant and low expression in M2 supernatant was observed. According to previous studies^{40,41}, secretion of IL-10 is thought to be one of the several feedback mechanisms after M1 polarization to limit excessive inflammatory responses. In fact, a more recent study from Ma et al. demonstrated the beneficial effects of adoptively transferred BM-M1 on liver scarring and regeneration in a mouse model of liver fibrosis²⁷. They reported that BM-M1 exhibit anti-fibrotic activity via recruitment of endogenous macrophages expressing MMP-2, 9, 13, increasing collagen degradation and hepatic growth factor (HGF) secretion, and inducing hepatocyte proliferation. They also found that NK cells migrated to the scar tissue leading to TRAIL-mediated apoptosis of HSCs and hampered fibrogenesis²⁷. Interestingly, in the present example, TRAIL expression was significantly higher in human iPSC-M1 macrophages compared to human iPSC-M2 macrophages.

Several clinical studies have tested the impact of macrophages on human patients with severe liver disease⁴². One such study from Forbes et al. on patients with cirrhosis tested the efficacy and safety of autologous macrophage therapy⁷. They showed several non-invasive measures of fibrosis improved following macrophage infusion, including transient elastography, serum enhanced liver fibrosis (ELF) score, and collagen turnover markers PRO-C3 and C3M; in conjunction, patients had a decreased model end stage liver disease (MELD) score one year after the trial. Since this study used autologous macrophages derived from an inflammatory, cirrhotic environment, a pro-inflammatory M1 polarized phenotype is assumed, yet antifibrotic therapeutic potential was achieved.

Taken together, both human iPSC-M1 and iPSC-M2 macrophages demonstrate signatures that contribute to the regenerative responses, including expression of collagenases and MMPs, and expression of phagocytic receptors and genes involved in anti-inflammatory activities including tissue repair and regeneration.

This example also investigated the survival and homing of human iPSC-derived macrophages to different organs including the liver. Previously, Haideri et al.¹² found that mESC-derived macrophages repopulated the Kupffer cell compartment of clodronate-treated mice more efficiently than BM-derived macrophages. This is of particular importance because recent studies have indicated that the human iPSC-derived macrophage phenotype is more comparable to tissue-resident (rather than monocyte-derived) macrophages^{24,43}. In the present example, true human iPSC-macrophages localized in the liver were not observed. This could be due to the intraperitoneal route of injection we used versus the intravenous route utilized by Haideri et al¹².

The use of a homogenous, standardized, and off-the-shelf product such as human iPSC-derived macrophages provides a potentially superior approach for treatment of hepatic diseases where liver transplantation is the only therapeutic option. The invention provides an efficient system for rapid generation of homogenous macrophage populations with anti-fibrotic effects from human iPSCs, a key step towards normalizing the use of these cells as a clinical therapeutic for human diseases including chronic liver diseases.

### Methods

### Induced Pluripotent Stem Cells Culture and Macrophage Differentiation

Human iPSCs derived from umbilical cord blood CD34⁺ cells^{15,44,45} were maintained as undifferentiated cells on matrigel-coated tissue culture flasks in mTeSR media (STEMCELL Technologies). Human iPSC-derived macrophages were generated according to previously described protocol⁴⁶ with some modifications. Briefly, 8,000 human iPSCs were single cell passaged and then aggregated into spin embryoid bodies (EBs) by centrifugation on ultra-low attachment 96-well u-bottom plates (Corning) with cytokines that are essential for hematopoietic progenitor development (APEL media containing 40 ng/ml SCF, 20 ng/ml VEGF, 20 ng/ml BMP4 and 10 μM Rock-Y). EBs were then manually transferred (20 EBs per well) onto 0.1% gelatin-coated 6-well plates containing differentiation medium I (X-VIVO™15 media (Lonza) supplemented with 1% penicillin-streptomycin (Gibco), 1% GlutaMAX (Gibco), 55 mM 2-Mercapnoethanol (Gibco), 50 ng/ml M-CSF and 25 ng/ml IL-3 (all from Peprotech)). Human iPSC-derived macrophage progenitor cells (iPSC-MPro) started to generate after 1-2 weeks. These cells were then collected and further differentiated to mature macrophages (human iPSC-derived macrophages) in differentiation medium II (X-VIVO™15 media supplemented with 1% penicillin-streptomycin, 1% GlutaMAX and 100 ng/ml M-CSF) for 7 days.

### Isolation of Peripheral Blood Monocyte and Differentiation to Macrophage.

Adult human blood was obtained from anonymous donors through the San Diego Blood Bank. Peripheral blood mononuclear cells (PBMC) were isolated by Ficoll-Paque Plus (GE Healthcare) density-gradient centrifugation from heparinized buffy coats. Monocytes (PB-monocyte) were isolated by CD14 positive selection using anti-CD14 magnetic beads (Miltenyi Biotec) according to manufacturer's instructions and then further differentiated to macrophages (PB-macrophages) in differentiation medium II for 7 days.

### Macrophage Polarization

Human iPSC-MPro or PB-monocytes were cultured in differentiation medium II for 7 days to differentiate to M0 (iPSC-M0 and PB-M0). M0 macrophages were then polarized to either M1 (iPSC-M1 and PB-M1) or M2 (iPSC-M2 and PB-M2) macrophages for 48 hours in differentiation medium II supplemented with different stimuli: 100 ng/mL LPS (Sigma) and 20 ng/mL IFN-g (R&D) for M1 polarization or 20 ng/ml IL-4 and 20 ng/ml IL-13 (R&D) for M2 polarization. The effect of activation was evaluated by quantifying changes in different phenotypic markers by flow cytometry, quantitative RT-PCR and RNA sequencing.

### Flow Cytometry

Single-cell suspensions were stained with antibodies listed in Table 1. Flow cytometry was performed on a BD LSR II or Acea Novocyte 3000, and the data were analyzed using Flowjo or NovoExpress software (Acea Biosciences).

Quantitative Real-Time PCR (qRT-PCR)

Total RNA was extracted from human iPSC and PB-derived macrophage populations using RNeasy Mini Kit (Qiagen) according to manufacturer's instructions. Complementary DNA (cDNA) was synthesized using iScript gDNA Clear cDNA Synthesis Kit (Bio-Rad) according to manufacturer's instruction. qRT-PCR was performed using the CFX384 Touch Real-Time PCR Detection System (Bio-Rad) and analyzed with CFX Manager™ Software (Bio-Rad). The TaqMan Gene Expression Assays (ThermoFisher Scientific) was used to detect gene expression. The ΔΔCt method was used with GAPDH to normalize cDNA levels and the “control” sample of each experiment was used as a calibrator to calculate relative change in gene expression. All data are presented as fold-change over the expression level of the calibrator.

### RNA-Seq and Analysis

All RNA sequencing analysis was performed by Novogene Corporation Inc. (Sacramento, CA, USA). All experiments were performed as 3 biological replicates. Differential expression analysis of two conditions/groups was done by using the DESeq2 R package, while the significant criterion was padj<0.05.

### Cytospin and Giemsa Staining

Cell suspension (10⁵) of different macrophage populations were loaded on slides using Cytospin (Thermo Shandon, Basingstoke, Hampshire, UK), spun at 500 rpm for 5 min and air-dried. Dried slides were stained with modified Giemsa (Polysciences, Inc.), according to manufacturer's instruction. Slides were mounted with round coverslips using Permount Mounting Medium (Fisher Chemical) and dried overnight. Images were taken using EVOS FLc (Life Technologies).

### Bead Phagocytosis Assay

For analysis of phagocytosis, different macrophage populations were incubated with carboxylate-modified red fluorescent latex beads with a mean diameter of 2 μm (L3030; Sigma-Aldrich) at a ratio of 1:10 for 2 hours. After repeated washing, the cells were analyzed by flow cytometry.

### Latex Bead Imaging

About 1.0×10⁶ macrophages were stained following CellTrace™ Violet Proliferation Kit (Invitrogen) protocol for suspension cells. Stained cells were then seeded on microscope cover glass (Fisherbrand) in 24-well plates coated with 0.1% gelatin and cultured overnight in differentiation medium II to allow re-attachment. On the following day, cells were incubated with latex beads as mentioned above. Cells were then fixed using 4% paraformaldehyde for 15 minutes at room temperature and washed with DPBS twice. Microscope cover glass was then mounted on glass slides using Permount Mounting Medium (Fisher Chemical) and dried overnight. Images were taken using EVOS FLc (Life Technologies).

### Cytokine/Chemokine Measurement

Supernatants from activated macrophage cultures were collected after 48 hours and frozen at −80° C. until assayed. Concentration of cytokines and chemokines were measured by Meso Scale Discovery (MSD), according to manufacturer's instructions.

### Animal Model of Liver Fibrosis

Eight-to ten-week-old Rag2^{-/-}γc^{-/-} mice (equal ratio of male and female) were purchased from the Jackson Laboratories and kept at the animal facility at the University of California, San Diego in accordance with IACUC regulations. Mice were divided into 4 groups and given increasing doses of CCl₄ in corn oil 47, or corn oil only as a control, over the course of 8 weeks. Doses of CCl₄ were slowly increased from 1:16 to 1:8 to 1:4. CCl₄ was administered via oral gavage twice weekly over this 8-week regimen. A timeline of the in vivo process is shown in FIG. 4A.

Groups of mice were therapeutically treated with either human iPSC-M1 or iPSC-M2 (5×10⁶, intraperitoneal injection) during the last 2 weeks (weeks 6 and 7) of the CCl₄ regimen. Injections were given 1 week apart, and mice were sacrificed 1 week after the second injection.

### Histology

Formalin-fixed, paraffin embedded murine livers were stained with H&E, Sirius Red, anti-αSMA (ab5694; Abcam), anti-desmin (Rb-9014-P0; Thermo Fisher Scientific), anti-F4/80 (14-4801-82; Thermo Fisher Scientific) and anti-CD68 (ab955, Abcam) antibodies. HRP conjugated secondary antibodies were utilized (anti-rabbit MP-7401 and anti-rat MP-7444; Vector Laboratories). For CD68, a mouse-on-mouse staining kit was used to block nonspecific host antibody-antigen interactions (Vector Laboratories, PK-2200). Signals were developed using DAB substrate (Vector Laboratories). Quantification of histological staining was done using ImageJ software.

Quantitative Real-Time PCR (qRT-PCR) for Fibrosis Assessment

Quantitative RT-PCR was performed using QuantStudio 3 system (Applied Biosystems). Total RNA was isolated from mouse livers using PureLink RNA Mini kit (Invitrogen). Expression levels of selected genes were measured. The data are shown as fold change of mRNA level expression compared to control (mean±SEM).

### Multiplex Cytokine Analysis On Tissue Homogenate

Levels of selected cytokines in liver homogenate were measured by the Rodent MAP 4.0-Mouse Sample Testing service provided by Ampersand Biosciences. Snap frozen total liver tissues were provided for analyses (https://www.ampersandbio.com/, Lake Clear, New York).

Human iPSC-Derived Macrophage Survival And Homing

To monitor the localization of human iPSC-derived macrophages, 3×10⁶ iPSC-M0 were labeled with PKH-26 red fluorescent dye (Sigma-Aldrich) and injected IP into eight-to ten-week-old uninjured Rag2^{-/-}γc^{-/-} mice 48. Mice were harvested at various time points over the course of 16 days (3, 5, 7, 10, 12, 14, 16 days post macrophage injection). Harvested organs including liver, kidney, and spleen, and peritoneal lavage were examined under an Olympus UV microscope (FIG. 10A).

To monitor the homing of human iPSC-macrophages into the livers of CCl₄-injured Rag2^{-/-}γc^{-/-} mice, a piggyBac plasmid expressing tdTomato (Vector Builder) was utilized to transfect human iPSCs using Amaxa™ Human Stem Cell Nucleofector™ Kit 1 (Lonza) following manufacturer's protocols. Transfected cells were cultured in presence of Puromycin (0.5 μg/ml) for 9 days until all cells expressed tdTomato and then they used to generate human iPSC-M2 macrophages as described above. 20×106 human iPSC-M2 were injected IP during week 7 of the CCl₄ regimen into Rag2^{-/-}γc^{-/-} mice. One week later, livers were harvested and evaluated for the presence of tdTomato-expressing human iPSC-M2 via histology.

These and other specific embodiments of the invention will be apparent to those skilled in the art upon a review of the present specification and non-limiting examples described herein.

### REFERENCES

1. Wang P, et al. Promising Therapy Candidates for Liver Fibrosis. jFront Physiol. 2016; 7: 47.
2. Kisseleva T, Brenner D A. The phenotypic fate and functional role for bone marrow-derived stem cells in liver fibrosis. J Hepatol. 2012; 56(4): 965-972.
3. Sze Y K, et al. Pediatric liver transplantation for metabolic liver disease: experience at King's College Hospital. Transplantation. 2009; 87(1): 87-93.
4. Thomas J A, et al. Macrophage therapy for murine liver fibrosis recruits host effector cells improving fibrosis, regeneration, and function. Hepatology. 2011; 53(6): 2003-2015.
5. Starkey Lewis P, et al. Alternatively activated macrophages promote resolution of necrosis following acute liver injury. J Hepatol. 2020; 73(2): 349-360.
6. Mohamadnejad M, et al. Randomized placebo-controlled trial of mesenchymal stem cell transplantation in decompensated cirrhosis. Liver Int. 2013; 33(10): 1490-1496.
7. Moroni F, et al. Safety profile of autologous macrophage therapy for liver cirrhosis. Nat Med. 2019; 25(10): 1560-1565.
8. Park S, et al. The therapeutic potential of induced hepatocyte-like cells generated by direct reprogramming on hepatic fibrosis. Stem Cell Res Ther. 2019; 10(1): 21.
9. Ding B S, et al. Divergent angiocrine signals from vascular niche balance liver regeneration and fibrosis. Nature. 2014; 505(7481): 97-102.
10. Nakamura T, et al. Significance and therapeutic potential of endothelial progenitor cell transplantation in a cirrhotic liver rat model. Gastroenterology. 2007; 133(1): 91-107.e101.
11. Watanabe Y, et al. Mesenchymal Stem Cells and Induced Bone Marrow-Derived Macrophages Synergistically Improve Liver Fibrosis in Mice. Stem Cells Transl Med. 2019; 8(3): 271-284.
12. Haideri S S, et al. Injection of embryonic stem cell derived macrophages ameliorates fibrosis in a murine model of liver injury. NPJ Regen Med. 2017; 2: 14.
13. Sica A, Mantovani A. Macrophage plasticity and polarization: in vivo veritas. J Clin Invest. 2012; 122(3): 787-795.
14. Duffield J S, et al. Selective depletion of macrophages reveals distinct, opposing roles during liver injury and repair. J Clin Invest. 2005; 115(1): 56-65.
15. Knorr D A, et al. Clinical-scale derivation of natural killer cells from human pluripotent stem cells for cancer therapy. Stem Cells Transl Med. 2013; 2(4): 274-283.
16. Suzuki M, et al. Tetraspanin CD9 negatively regulates lipopolysaccharide-induced macrophage activation and lung inflammation. J Immunol. 2009; 182(10): 6485-6493.
17. Gabay C, Palmer G. Mutations in the ILIRN locus lead to autoinflammation. Nat Rev Rheumatol. 2009; 5(9): 480-482.
18. Peshkova I O, et al. IL-27R signaling controls myeloid cells accumulation and antigen-presentation in atherosclerosis. Sci Rep. 2017; 7(1): 2255.
19. Junkins R D, et al. Regulator of calcineurin 1 suppresses inflammation during respiratory tract infections. J Immunol. 2013; 190(10): 5178-5186.
20. PrabhuDas M R, et al. A Consensus Definitive Classification of Scavenger Receptors and Their Roles in Health and Disease. J Immunol. 2017; 198(10): 3775-3789.
21. Li B, et al. The melanoma-associated transmembrane glycoprotein Gpnmb controls trafficking of cellular debris for degradation and is essential for tissue repair. Faseb j. 2010; 24(12): 4767-4781.
22. Chung H, et al. Minireview: Syndecans and their crucial roles during tissue regeneration. FEBS Lett. 2016; 590(15): 2408-2417.
23. Alexopoulou A N, et al. Syndecans in wound healing, inflammation and vascular biology. Int J Biochem Cell Biol. 2007; 39(3): 505-528.
24. Buchrieser J, et al. Human Induced Pluripotent Stem Cell-Derived Macrophages Share Ontogeny with MYB-Independent Tissue-Resident Macrophages. Stem Cell Reports. 2017; 8(2): 334-345.
25. Zhang H, et al. Functional analysis and transcriptomic profiling of iPSC-derived macrophages and their application in modeling Mendelian disease. Circ Res. 2015; 117(1): 17-28.
26. Kisseleva T, Brenner D A. Role of hepatic stellate cells in fibrogenesis and the reversal of fibrosis. J Gastroenterol Hepatol. 2007; 22 Suppl 1: S73-78.
27. Ma P F, et al. Cytotherapy with M1-polarized macrophages ameliorates liver fibrosis by modulating immune microenvironment in mice. J Hepatol. 2017; 67(4): 770-779.
28. Guillot A, et al. Interleukins-17 and 27 promote liver regeneration by sequentially inducing progenitor cell expansion and differentiation. Hepatol Commun. 2018; 2(3): 329-343.
29. Terai S, Tsuchiya A. Status of and candidates for cell therapy in liver cirrhosis: overcoming the “point of no return” in advanced liver cirrhosis. J Gastroenterol. 2017; 52(2): 129-140.
30. Shiraishi M, et al. Alternatively activated macrophages determine repair of the infarcted adult murine heart. J Clin Invest. 2016; 126(6): 2151-2166.
31. Watanabe T, et al. Gene expression of interstitial collagenase in both progressive and recovery phase of rat liver fibrosis induced by carbon tetrachloride. J Hepatol. 2000; 33(2): 224-235.
32. Fallowfield J A, et al. Scar-associated macrophages are a major source of hepatic matrix metalloproteinase-13 and facilitate the resolution of murine hepatic fibrosis. J Immunol. 2007; 178(8): 5288-5295.
33. Uchinami H, et al. Loss of MMP 13 attenuates murine hepatic injury and fibrosis during cholestasis. Hepatology. 2006; 44(2): 420-429.
34. Iredale J P. Hepatic stellate cell behavior during resolution of liver injury. Semin Liver Dis. 2001; 21(3): 427-436.
35. Popov Y, et al. Macrophage-mediated phagocytosis of apoptotic cholangiocytes contributes to reversal of experimental biliary fibrosis. Am J Physiol Gastrointest Liver Physiol. 2010; 298(3): G323-334.
36. Bataller R, Brenner D A. Liver fibrosis. J Clin Invest. 2005; 115(2): 209-218.
37. Triantafyllou E, et al. MerTK expressing hepatic macrophages promote the resolution of inflammation in acute liver failure. Gut. 2018; 67(2): 333-347.
38. Dehn S, Thorp E B. Myeloid receptor CD36 is required for early phagocytosis of myocardial infarcts and induction of Nr4a1-dependent mechanisms of cardiac repair. Faseb j. 2018; 32(1): 254-264.
39. Coelho I D N, Barros A, Macedo M P, Penha-Gonçalves C. Trem-2 promotes emergence of restorative macrophages and endothelial cells during recovery from hepatic tissue damage. bioRxiv. 2019.
40. Cao X, et al. Differentiation and Functional Comparison of Monocytes and Macrophages from hiPSCs with Peripheral Blood Derivatives. Stem Cell Reports. 2019; 12(6): 1282-1297.
41. Stanley A C, et al. Recycling endosome-dependent and-independent mechanisms for IL-10 secretion in LPS-activated macrophages. J Leukoc Biol. 2012; 92(6): 1227-1239.
42. Moore J K, et al. Phenotypic and functional characterization of macrophages with therapeutic potential generated from human cirrhotic monocytes in a cohort study. Cytotherapy. 2015; 17(11): 1604-1616.
43. Haenseler W, et al. A Highly Efficient Human Pluripotent Stem Cell Microglia Model Displays a Neuronal-Co-culture-Specific Expression Profile and Inflammatory Response. Stem Cell Reports. 2017; 8(6): 1727-1742.
44. Hermanson D L, et al. Induced Pluripotent Stem Cell-Derived Natural Killer Cells for Treatment of Ovarian Cancer. Stem Cells. 2016; 34(1): 93-101.
45. Li Y, et al. Human iPSC-Derived Natural Killer Cells Engineered with Chimeric Antigen Receptors Enhance Anti-tumor Activity. Cell Stem Cell. 2018; 23(2): 181-192.e185.
46. van Wilgenburg B, et al. Efficient, long term production of monocyte-derived macrophages from human pluripotent stem cells under partly-defined and fully-defined conditions. PLOS One. 2013; 8(8): e71098.
47. Kisseleva T, et al. Myofibroblasts revert to an inactive phenotype during regression of liver fibrosis. Proc Natl Acad Sci U S A. 2012; 109(24): 9448-9453.
48. Liu X, et al. Identification of Lineage-Specific Transcription Factors That Prevent Activation of Hepatic Stellate Cells and Promote Fibrosis Resolution. Gastroenterology. 2020; 158(6): 1728-1744.e1714.

What is claimed is:

1. A method of treatment of damaged liver tissue, comprising administering to a subject in need thereof an effective amount of a composition comprising a plurality of macrophages derived from human induced pluripotent stem cells (iPSCs), wherein the macrophages are polarized to a pro-inflammatory M1 phenotype and/or an anti-inflammatory M2 phenotype.

2. The method of claim 1, wherein the subject has liver fibrosis.

3. The method of claim 1, wherein the administration reduces fibrogenic gene expression and liver disease associated histological markers.

4. The method of claim 1, wherein the M1 macrophages express elevated CD80, TNF-α and IL-6.

5. The method of claim 1, wherein the M2 macrophages express elevated CD206, CCL17, and CCL22.

6. A composition comprising a plurality of macrophages derived from human induced pluripotent stem cells (iPSCs), wherein the macrophages are polarized to a pro-inflammatory M1 phenotype and/or an anti-inflammatory M2 phenotype.

7. The method of claim 1, wherein the M1 macrophages express elevated CD80, TNF-α and IL-6.

8. The method of claim 1, wherein the M2 macrophages express elevated CD206, CCL17, and CCL22.

9. A method for the manufacture of a cellular composition comprising deriving a plurality of macrophages from human induced pluripotent stem cells (iPSCs), and polarizing the macrophages to a pro-inflammatory M1 phenotype and/or an anti-inflammatory M2 phenotype, thereby manufacturing the cellular composition.

10. The method of claim 9, wherein the macrophages are polarized to pro-inflammatory M1 in presence of LPS+IFN-γ.

11. The method of claim 10, wherein the M1 macrophages express elevated CD80, TNF-α and IL-6.

12. The method of claim 9, wherein the macrophages are polarized to anti-inflammatory M2 phenotypes in presence of IL-4+IL-13.

13. The method of claim 12, wherein the M2 macrophages express elevated CD206, CCL17, and CCL22.

14. A pharmaceutically acceptable composition comprising a plurality of macrophages derived from human induced pluripotent stem cells (iPSCs), wherein the macrophages are polarized to a pro-inflammatory M1 phenotype and/or an anti-inflammatory M2 phenotype.

15. The composition of claim 14, wherein the macrophages have been polarized to pro-inflammatory M1 in presence of LPS+IFN-γ so as to express elevated CD80, TNF-α and IL-6.

16. The composition of claim 14, wherein the macrophages have been polarized to anti-inflammatory M2 phenotypes in presence of IL-4+IL-13 so as to express elevated CD206, CCL17, and CCL22.
